(19) United States
(12) Patent Application Publication
(10) Pub. No.: US 2025/0313811 A1
(43) Pub. Date: Oct. 9, 2025

(54) PROCESS FOR PREPARATION AND CRYOPRESERVATION OF DENTAL PULP FROM DEFINITIVE TEETH AND PRODUCTS THEREOF BASED ON ISOLATED MESENCHYMAL STEM CELLS

(71) Applicant: ICETA - INSTITUTO DE CIÊNCIAS, TECNOLOGIAS E AGROAMBIENTE DA UNIVERSIDADE DO PORTO, Porto (PT)

(72) Inventor: Ana MAURICIO, Porto (PT)

(21) Appl. No.: 19/117,185

(22) PCT Filed: May 4, 2023
(86) PCT No.: PCT/IB2023/054631

§ 371 Date: Mar. 31, 2025

(30) Foreign Application Priority Data
May 24, 2022 (PT) 117999

(51) Int. Cl. C12N 5/00; C12N 5/0775

(52) U.S. Cl. CPC C12N 5/562; C12N 5/0664; C12N 5/524; C12N 5/54

(57) ABSTRACT

The present invention relates to a process for preparation and cryopreservation of dental pulp teeth and products thereof resulting in innovative cellular systems useful for therapeutic application based on the mesenchymal stem cells, so called dental pulp stem cells (DPSCs).

The objective of this invention is to provide the most adequate cellular isolates from dental pulp tissue from a tooth of a human subject. Fast expanding populations of DPSCs can be obtained, while maintaining their chromosomal stability, and determined to present the phenotypical and functional characteristics desired of such populations.

In another aspect, the present invention provides a novel and simplified method increasing thSpece viability of the dental tissue during the storing and banking. Also, the isolation of DPSCs from these teeth is improved and herein disclosed.

Therefore, the present invention is in the field of cell-based therapies, regenerative medicine, and optimized processes for obtaining the desired cell-isolates.

### TECHNICAL FIELD OF INVENTION

The present invention relates to a process for preparation and cryopreservation of dental pulp from teeth and products thereof resulting in innovative cellular systems useful for therapeutic application based on the mesenchymal stem cells, so called dental pulp stem cells (DPSCs).

The objective of this invention is to provide the most adequate cellular isolates from dental pulp tissue from a tooth of a human subject. Fast expanding populations of DPSCs can be obtained, while maintaining their chromosomal stability, and determined to present the phenotypical and functional characteristics desired of such populations.

In another aspect, the present invention provides a novel and simplified method increasing the viability of the dental tissue during the storing and banking. Also, the isolation of DPSCs from these teeth is improved and herein disclosed.

Therefore, the present invention is in the field of cell-based therapies, regenerative medicine, and optimized processes for obtaining the desired cell-isolates.

### PRIOR ART

In the last decades considerable attention has been directed by the scientific community to the derivation of cells, in particular stem cells maintaining the ability to differentiate into specific tissue cells. This interest has increased by a strong need and desire to develop novel tissue and cell replacement approaches to heal the ailing body and re-establish cells and functions of afflicted and damaged tissues and parts of the body.

These cells have the capacity to self-renew and to develop into different specialized cells. Mammalian stem cells are categorized into two general types, embryonic stem cells found during early embryonic development, and adult stem cells found in tissues of the organism at later stages and throughout life.

Stem cells can be classified according to their differentiation potential as: totipotent, pluripotent and multipotent.

Totipotent stem cells can grow and form a complete organism, forming both embryonic components (such as the three germ layers, the germline and the tissues which will give rise to the yolk sac, for example) and extraembryonic components (such as the placenta). In other words, they can form all the cell types.

Multipotent stem cells are those which can only generate cells of their own germ layer or germline of origin, for example: since a bone marrow mesenchymal stem cell has a mesodermal nature, it will give rise to cells of that layer such as myocytes, adipocytes or osteocytes, among others.

Pluripotent stem cells cannot form a complete organism, but they can differentiate into cells from the three germ layers: (a) ectoderm, which is the origin of the nervous system, the respiratory system, upper digestive tract (stomodeum), the epidermis and its adnexa (hair and nails) and the mammary glands; (b) endoderm, which is the origin of the intestine, the liver, the pancreas, the lungs and most of the internal organs; and (c) mesoderm, which is the origin of the skeletal system, the muscles and the circulatory and reproductive systems. They can also form any other type of cell from the germ and the yolk sac.

These pluripotent cells have the capacity to differentiate into such a large number of tissues makes them especially interesting for the design of new therapies in general and of regenerative therapies in particular. Currently, pluripotent stem cells in adult individuals are mainly obtained from bone marrow.

However, adult stem cells have limited capacity for differentiation in comparison with pluripotent embryonic stem cells and can usually differentiate to form only specific cell types of their tissue of origin. In contrast to embryonic stem cells, adult stem cells also known as mesenchymal stem cells (MSCs) are not able to build a whole organism.

Besides the inherent difficulty and challenge for using MSCs in therapy due to their differentiation capacity dependence on suitable isolation and culture conditions, they are found only in infinitesimal numbers, and it is extremely difficult to isolate them in useful amounts. Moreover, their propagative capacity is relatively low, and they may contain various DNA aberrations.

The main sources of MSCs currently used in medicine are the bone marrow and the umbilical cord. Alternative sources for the isolation of highly pluripotent and homogeneous stem cells are required, which will significantly increase the treatment efficacy of several diseases.

Presently, it is recognized that teeth are a readily accessible source for obtaining MSCs useful for tissue regeneration and repair. Similar to other organs in the human body, the teeth and their surrounding tissues are composed by mixed populations of cells, which include multipotent MSCs/pericytes, progenitor and differentiated cells.

In recent years, a large body of scientific literature has provided evidence that dental pulp represents an easily accessible source of stem cells which can be easily cultured. Moreover, similarly to other stem cell types, DPSCs have shown broad differentiation potential, suitable for the development—still in its infancy—of therapeutic applications for the regeneration of bone, cornea, spinal cord injuries, post-ischemic cerebral tissue among others. The above has increased the interest in long term dental stem cell banking.

To conduct stem cell therapies significant in vitro, expansion of stem cells is necessary in order to generate sufficient quantities of these cells to treat human disease. At the present time, there is no known reliable way to efficiently generate large numbers of relatively pure dental pulp stem cells populations in culture.

One of the reasons for this is that the regenerative potential diminishes with age, and this has been ascribed to functional impairments of adult stem cells.

Additionally, is well known that cells in culture undergo senescence after a certain number of cell divisions whereby the cells enlarge and finally stop proliferation. Aging and replicative senescence have related effects on human stem and progenitor cells making the scale-up production and expansion a considerable challenge.

EP1305400A1 discloses a culture of isolated adult human dental pulp stem cells and a method for use for regenerating or producing a dentin/pulp tissue, by contacting a cell from a culture of isolated adult human dental pulp stem cells with hydroxyapatite/tricalcium phosphate. The cells can be used for transplanting into a mammal. However, this method only uses stem cell that produces odontoblast-specific dentin sialoprotein. Thus, the isolated thereof is only useful for treating and restorative teeth purposes.

EP2456863A1 discloses pluripotent stem cells (DPPSCs) obtained from dental pulp of patients of different ages and cultures of such cells.

US20200339954A1 discloses compositions of multifunctional immature dental pulp stem cells (IDPSCs), methods for generating clinically useful amount of IDPSCs from the dental pulp (DP) of a patient or single donor at early passages with minimal risk of losing their “stemness” in order to use the stem cells thus obtained in stem cell therapy, and clinical and aesthetic use of IDPSC multi-lineage oriented therapeutic compositions to prevent and treat degenerative diseases and medicinal and aesthetic symptoms.

Considering the multitude of cellular sources reported, research focused on the selection of suitable cellular sources, and considering the technical and biological requirements imposed for the effective therapeutic application of the known systems there is a clear limitation in the availability not only stem-cells sources but even more in suitable stem-cells that can be used reliably, consistently, and accurately in cell-based therapies and research.

The invention disclosed herein discloses a method to increase the source and quality of DPSCs, whilst reducing the complexity associated to the isolation of these cells, with reduced costs associated to the production of resulting isolates.

### DESCRIPTION OF THE FIGURES

FIG. 1. Presents micro-holes made in all teeth before cryopreservation. These holes (4-8) were drilled in a premolar tooth, in the area of the tooth neck for extraction of the dental pulp after thawing. The additional 4-8 holes should not penetrate the dental pulp cavity.

FIG. 2. Presents the extraction of dental pulp of a tooth. With the aid of two clamps, one clamp applied at the level of the crown of the tooth, and another applied at the level of the roots, and with reverse rotation movements, the tooth was opened, and the dental pulp was exposed. A premolar tooth was opened for extraction of cryopreserved dental pulp and after rapid thawing procedure in a water bath at 37° C.

FIG. 3. Presents an isolate and expansion of DPSCs. An explant of cryopreserved dental pulp shows the isolation and expansion of DPSCs, 3 days after thawing and the beginning of cell culture. Image obtained in an inverted microscope with 200× magnification.

### DESCRIPTION OF THE INVENTION

The present invention relates to a process for preparation and cryopreservation of dental pulp from a tooth of a human subject, products thereof resulting in innovative cellular systems useful for therapeutic application concerning based on the mesenchymal stem cells, so called dental pulp stem cells (DPSCs).

Different type of teeth, such as incisor, canine, premolar and molar teeth, of the mandibular arch, maxillary arch and included teeth, either from adults (definitive teeth) and children (deciduous teeth) can be used.

Dental pulp is subject to cryopreservation and posterior isolation of DPSCs, when thawed. Only healthy teeth, such as the ones extracted by reasons for orthodontic correction, teeth with no visible pathology that affects the dental pulp (tooth decay, abscesses, periodontal disease) can be used, to ensure that led to the cryopreservation of healthy dental pulp, which, after rapid thawing, allowed the isolation and in vitro expansion of DPSCs.

In order to ensure the viability of the dental pulp and to limit the microbiological contamination of the collected teeth, their transport from the place of removal to the laboratory must be refrigerated and not exceeding 72 hours.

The washing of the teeth must be done before drilling the holes in the tooth and the cryopreservation process. An initial cleaning of the tooth should be carried out with the use of sterile compresses soaked in 70% ethyl alcohol (V/V) until total removal of food debris, blood and gums that may result from the tooth extraction process. This procedure must be repeated as many times as necessary until all dental surfaces (dental crown, contact surfaces and roots) are clean and free of residues.

Then, the tooth should be immersed in a sterile phosphate buffer solution (DPBS) suitable to be in contact with samples of human origin.

To proceed with disinfection, the tooth must be immersed in a 70% ethyl alcohol (V/V) solution for 30 seconds. After this disinfection step, the tooth must be washed again with sterile phosphate buffer solution (DPBS).

After the washing and disinfection process, the necessary holes must be made in the tooth for the penetration of the cryopreservation solution. 1-2 transverse and diametrically opposed holes should be made in the neck of incisor and canine teeth, or 2-4 transverse and diametrically opposed holes in the neck of premolar or molar teeth, in order to create channels up to the cavity of the dental pulp, without damaging the dental pulp. During drilling, the drill must be cooled with refrigerated sterile saline solution in order to avoid overheating which could damage the dental pulp and compromise the viability of the mesenchymal stem cells of the dental pulp.

After drilling the holes, a sterile file should be used to collect the dental pulp sample for microbiological analysis. The file must be inoculated in a nutritive culture medium to control microbiological quality.

Microbiological analysis for aerobic and anaerobic microorganisms, identification and antibiogram should be performed. To collect dental pulp samples, only one sterile file (K file 10 or 15 with 25 mm) is used per analysed tooth. The files are introduced to the maximum possible length in all the drilled holes and then they are totally immersed in the nutrient culture medium suitable for microbiological analysis (heart-brain broth culture medium, IVD).

The collection tubes dental samples for with pulp microbiological analysis are hermetically sealed and kept at room temperature and were always sent to the analysis laboratory on the day of collection of dental pulp samples for analysis.

The volume of cryopreservation solution is calculated so that each tooth is cryopreserved in a sterile cryovial of 10 ml capacity. The proportions of the cryopreservation solution are shown in Table 1 and each cryovial should be filled with 7.7 ml of cryopreservation solution.

TABLE 1
Components of the cryopreservation solution and their
proportion for 7.7 ml of cryopreservation solution.

| Cryopreservation solution | Volume for 7.7 ml |
|---|---|
| Culture medium xeno-free | 5.6 ml |
| Human Serum Off the Clot Type AB | 1.4 ml |
| Dimethyl Sulfoxide (DMSO) | 0.7 ml |

The drilled tooth should be immersed the cryopreservation solution (Table 1) and then transferred to the cryopreservation tank well.

The isolation of DPSCs is performed by explant procedure after thawing of the cryopreserved teeth.

The processed and thawed teeth are then transferred to a sterile container containing isolation and expansion culture medium (called expansion culture medium) (Table 2).

TABLE 2
Components of the expansion culture medium and their
proportion to 100 ml.

| Maintenance culture medium | 100 ml solution |
|---|---|
| Culture medium xeno-free | 80% (3.5 ml), 80 ml |
| Human Serum Off the Clot, Type | 20% (3.5 ml), 20 ml |
| AB, without heparin | |

An antibiotic and anti-mycotic solution comprising 100 U/ml penicillin, 100 mg/ml streptomycin and 250 μg/ml of amphotericin B is added to the expansion culture medium (0.1 ml).

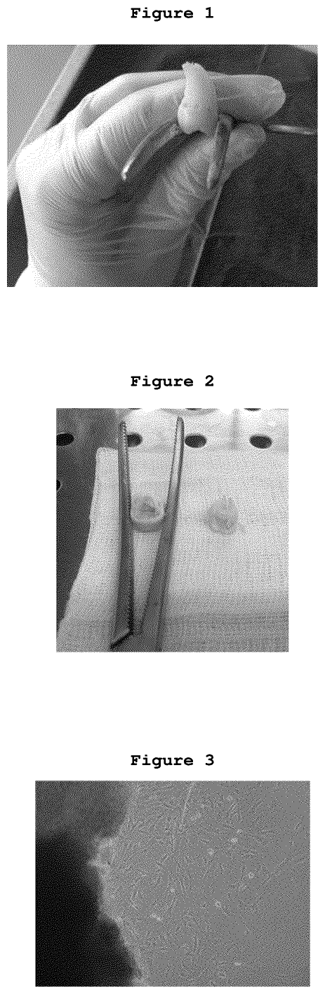

Micro-holes were made in all teeth before cryopreservation, as shown in FIG. 1. These holes (4-8) were drilled in a premolar tooth, in the area of the tooth neck for extraction of the dental pulp after thawing. The additional 4-8 holes should not penetrate the dental pulp cavity.

Then, with the aid of two clamps, one clamp applied at the level of the crown of the tooth, and another applied at the level of the roots, and with reverse rotation movements, the tooth was opened, and the dental pulp is exposed. A premolar tooth was opened for extraction of cryopreserved dental pulp and after rapid thawing procedure in a water bath at 37° C., as shown in FIG. 2.

The dental pulp of all teeth is extracted using sterile forceps and cut into 1-2 mm fragments using a sterile scalpel. The dental pulp fragments from each tooth are transferred to T75 flasks or Petri dishes, in which 10 ml or 2 ml of expansion culture medium are previously placed.

The DPSCs are allowed to grow in a CO2 incubator at 37° C. for 3 days and are maintained for the following weeks (twice a week the culture medium is changed) and after 7-10 days it is possible to obtain the isolation and expansion of DPSCs with desired confluence. Explant of a cryopreserved dental pulp demonstrating the isolation and expansion of DPSCs, 3 days after thawing and the beginning of cell culture is shown in FIG. 3. The image was obtained by using an inverted microscope with 200× magnification.

DPSCs thus obtained are characterized by high proliferative capacity and plasticity and can give rise in vitro to cell lineages both of mesenchymal origin, such as osteoblasts, adipocytes, chondrocytes, and striated muscle cells and of non-mesenchymal origin, such as melanocytes.

When DPSCs are banked in view of future research or potential therapeutic applications, they are usually cryopreserved after tooth mechanical fracture and in vitro expansion.

The latter procedures generate significant workload and require resources that must be made available upfront for all banked teeth, regardless of the number actually used at the time of future requirement.

A more rational and less expensive approach to long term banking can be to simplify the initial cryopreservation procedure, delaying complex processing procedures to later phases of actual use, such as the cryopreservation method herein disclosed.

The product is completely validated in terms of in vitro cytocompatibility, and in vivo biocompatibility. The processing of permanent and deciduous teeth (incisor, canine, premolar and molar teeth of the mandibular, maxillary arches and included teeth for permanent dentition, incisor, canine and premolar teeth of the mandibular, maxillary arches for deciduous dentition) the for cryopreservation of the respective dental pulp was already validated in order to ensure that the pulp tissue after thawing has viability for the isolation and in vitro expansion of mesenchymal stem cells, called mesenchymal stem cells of the dental pulp or DPSCs, for eventual application autologous or allogeneic clinic. The clinical application of DPSCs will be in the scope of Regenerative Medicine, namely in bone, nervous, vascular, musculoskeletal regeneration, among others.

Teeth extraction must be carried out by a dentist, stemmatologist or a professional in this area.

The teeth must be cryopreserved in liquid nitrogen (gas phase) and subjected to rapid thawing in a water bath at 37° C., for isolation and expansion of DPSCs.

For in vitro validation of the process/protocol, the DPSCs isolated from the cryopreserved explants, were in vitro characterized according to the parameters defined by the International Society for Cellular Therapies (ISCT), namely: when in proliferation and multiplication, they adhere to plastic surfaces; exhibit MSCs surface markers, such as CD44, CD73, CD90, CD105, CD117; and have the ability to differentiate in at least 3 distinct lines (when exposed to suitable culture media they can give rise to bone tissue cells-osteogenic differentiation, cartilage differentiation-chondrogenic differentiation, adipose tissue—adipogenic differentiation, muscle tissue—differentiation myogenic and neuroglial cells—neurogenic differentiation).

In addition, tests were carried out in order to verify the chromosomal stability during cell cultures for isolation and expansion, the biocompatibility of DPSCs when applied to animal models (rat experimental model of axonotmesis and neurotmesis lesions) and the production capacity of growth factors.

### EXAMPLES

### Example 1. Defrosting of the Cryopreserved Tooth

To thaw the cryopreserved teeth and the respective dental pulps, the cryotubes were placed directly in a water bath at 37° C. and then the tooth should be immediately transferred to a sterile container containing StemPro®MSC SFM Xeno-Free culture medium supplemented with 20% Human Serum Off the Clot (Type AB, CAPRICORN, without heparin), 0.1% gentamicin 10 mg/ml, 0.1% penicillin and streptomycin and fungizone amphotericin B (called expansion culture medium).

### Example 2. Isolation and In Vitro Expansion of DPSCs from Thawed Dental Pulp Units

In a sterile environment (laminar flow chamber), using the dental drill (Surgical Engine W&H Implantmed) and the respective drills of 0.12-0.14 mm in diameter (Komet FG 801-012 or FG 801-014) were performed 4-8 micro-holes, in the intervals of the micro-channels made during processing, at the level of the tooth neck and without reaching the cavity of the dental pulp.

With the aid of two clamps (one clamp applied at the level of the crown of the tooth and another applied at the level of the roots) and with reverse rotation movements, the tooth was opened for the exposure of the dental pulp.

The dental pulp was extracted using sterile forceps and cut into 1-2 mm fragments using a sterile scalpel. The dental pulp fragments were transferred to T75 flasks or Petri dishes, in which 10 ml or 2 ml of expansion culture medium was previously placed, respectively. The cells were allowed to grow in a CO₂ Incubator at 37° C. for 3 running days. It must be maintained during subsequent weeks (twice a week the culture medium was changed) so that, after 7-10 days, it was possible to obtain the desired confluence.

The isolation and expansion of DPSCs allowed to evaluate the viability of the dental pulp after the tooth has been processed and cryopreserved. The units that demonstrate the isolation and expansion of the DPSCs were photographed and validated according to morphological analysis in an inverted microscope, at a magnification of 200-400×.

The isolated DPSCs and expanded to P3-P5 by dental pulp fragment explant technique, were subjected to a complete in vitro characterization, by immunocytochemistry, flow cytometry, secretome, karyotype, RT-PCR and differentiation in at least three or four cell lines.

The in vivo application in the experimental rat model (lesions of axonotmesis and neurotmesis) of isolated DPSCs and expanded to P3-P5 was performed.

### Example 3. In Vitro Characterization of Isolated and Expanded—Immunocytochemistry with NANOG, Stro-1, c-kit, CD31 and Vimentin Markers

In passage 3 (P3), DPSCs were trypsinized, washed and resuspended in Shandon™ Cytoblock™ Cell Block Preparation System (Thermo Scientific, USA) at a minimum concentration of 1×10⁵ cells/ml and processed for immunocytochemical analysis with the markers Nanog, Stro-1, c-kit, CD31 and vimentin. Antigen recovery was carried out in dewaxed sections, by immersion in citrate buffer (10 mM, pH 6.0) in a pressure cooker for 3 minutes. The Novolink™ Max-Polymer detection system (Novocastra, UK) was used to view the preparation, according to the manufacturer's instructions and the sequence of monoclonal antisera used were: vimentin (clone V9, Dako) diluted to 1:500; CD117 (c-Kit) (A4502; DakoCytomation) diluted 1:450; CD31 (clone JC70A, Dako) diluted 1:50 and NANOG-1 (clone MAB, ABGent) diluted 1:50. The slides of the sections dewaxed with the marked DPSCs were observed under a vertical microscope at 200× or 400× magnifications.

### Example 4. Immunocytochemistry with the GFAP, GAP-43 and NeuN Markers of Undifferentiated DPSCs and After Differentiation in Neuroglial Cells

In passage 3 (P3), the DPSCs were trypsinized, washed and resuspended in expansion culture medium at a concentration of 1×10⁵ cells/ml. The DPSCs were fixed with paraformaldehyde at 4° C. for 15 min and then washed with distilled water before permeabilization in 0.5% Triton-X100. Non-specific binding was blocked with blocking solution (PBS containing 1% bovine serum albumin (BSA)) for 1 hour at room temperature. The DPSCs were then incubated for 2 hours at room temperature, with primary antibodies anti-growth-associated protein-43 (GAP-43, 1:200), rabbit anti-glial fibrillary acid protein (GFAP, 1:500), rabbit and mouse neuronal anti-nucleus (NeuN, 1:100). After washing, 15 minutes were incubated with secondary goat anti-mouse IgG In passage antibody 3 (P3), the DPSCs were trypsinized, washed and resuspended in expansion culture medium at a concentration of 1×10⁵ cells/ml. The DPSCs were fixed with paraformaldehyde at 4° C. for 15 min and then washed with distilled water before permeabilization in 0.5% Triton-X100. Non-specific binding was blocked with blocking solution (PBS containing 1% bovine serum albumin (BSA)) for 1 hour at room temperature. The DPSCs were then incubated for 2 hours at room temperature, with primary antibodies anti-growth-associated protein-43 (GAP-43, 1:200), rabbit anti-glial fibrillary acid protein (GFAP, 1:500), rabbit and mouse neuronal anti-nucleus (NeuN, 1:100). After washing, 15 minutes were incubated with secondary goat anti-mouse IgG antibody and goat anti-rabbit IgG antibody. After several washes in PBS, they were incubated with horseradish peroxidase (HRP) —treptavidin for 10 min. DAB (diaminobenzidine) was the chromogen used.

### Example 5. Flow cytometry with the CD34, CD 45, CD73, CD90, CD105 markers of the isolated DPSCs and expanded to P3

Flow cytometry was performed on a FACSCalibur®, BD Biosciences device with the DPSCs suspended in isotonic medium, following the manufacturer's instructions. Flow cytometric analysis was performed with the following antibodies and their isotypes (BioLegend): anti-human PE CD105 (eBioScience); Anti-human APC CD73; Anti-human PE CD90; PerCP/Cy5.5 anti-human CD45: FITC anti-human CD34; PerCP/Cy5.5 anti-human CD14; Pacific Blue anti-human CD19 and pacific-blue anti-human HLA-DR.

### Example 6. Secretome of the DPSCs Isolated and Expanded to P3-P5 by Multiplexing LASER Bead Technology

DPSCs isolated and expanded to P3-P5 were characterized with respect to the secretome (secretion of growth factors, cytokines, chemokines and inflammatory mediators), after 24 h and 48 h in basal culture medium (not supplemented with Human Serum Off the Clot, Type AB, CAPRICORN). The samples were analysed using Multiplexing LASER Bead Technology (Eve Technologies, Canada) to detect and quantify growth factors, cytokines, chemokines and inflammatory mediators.

### Example 7. Cytogenetic Analysis of Isolated and Expanded DPSCs up to P3-P5 (Karyotype in Expansion Phase)

The DPSCs isolated from the cryopreserved dental pulp explants were studied for cytogenetic analysis in P3-P5. When the confluence of the cell culture was verified, the culture medium was changed and supplemented with a 4 μg/ml colcemide solution. After 4 hours, the DPSCs were collected and suspended in 8 ml of a 0.075M solution of KCI supplemented with foetal bovine serum (SFB). Then, the suspension was incubated at 37° C. for 35 minutes. After centrifugation (1500 rpm), the fixative composed of methanol and glacial acetic acid was added to the cell pellet 8 ml, and then further centrifugations (normally 4). After the last centrifugation, the cell suspension of DPSCs was dispersed on glass slides and stained with Giemsa for cytogenetic analysis of the karyotype. With cytogenetic analysis, tooth processing and cryopreservation of the respective dental pulp was validated, in terms of obtaining DPSCs from explants, which have chromosomal stability.

### Example 8. RT-PCR Analysis: Gene Expression of β-Actin, GAPDH, c-kit, Oct-4, NANOG and ALP from isolated and expanded DPSCs to P3-P5

Using techniques based on DNA detection, particularly RT-PCR (reverse transcriptase reaction, followed by polymerase chain reaction, Reverse transcriptase-Polymerase chain reaction, maintaining the international nomenclature) and qPCR (quantitative PCR), analysis of the expression of six genes, two ‘housekeeping genes’ (β-actin and GAPDH) and four genes that encode stem cell pluripotency markers (c-kit, Oct-4, NANOG and ALP). Regarding the selected target genes, the sequence of the Nanog gene encodes a transcription factor present in embryonic stem cells, involved in the maintenance of its pluripotency; Oct-4 is also a transcription factor involved in the self-renewal process of undifferentiated embryonic stem cells and, for this reason, it is often used as a marker of the undifferentiated character of stem populations; similarly, the c-kit sequence, also referred to as CD117, is also a stem cell pluripotency marker; the genetic Using techniques based on DNA detection, particularly RT-PCR (reverse transcriptase reaction, followed by polymerase chain reaction, Reverse transcriptase-Polymerase chain reaction, maintaining the international nomenclature) and qPCR (quantitative PCR), analysis of the expression of six genes, two ‘housekeeping genes’ (β-actin and GAPDH) and four genes that encode stem cell pluripotency markers (c-kit, Oct-4, NANOG and ALP). Regarding the selected target genes, the sequence of the Nanog gene encodes a transcription factor present in embryonic stem cells, involved in the maintenance of its pluripotency; Oct-4 is also a transcription factor involved in the self-renewal process of undifferentiated embryonic stem cells and, for this reason, it is often used as a marker of the undifferentiated character of stem populations; similarly, the c-kit sequence, also referred to as CD117, is also a stem cell pluripotency marker; the genetic sequence ALP (alkaline phosphatase-alkaline phosphatase maintaining the international nomenclature) encodes a hydrolase involved in several dephosphorylation processes, being expressed in the membrane of undifferentiated pluripotent cells and, as such, also used as a stem cell marker.

RT-PCR and qPCR of specific genes typically expressed by pluripotent and multipotent stem cells such as DPSCs was performed.

Dental pulp-derived mesenchymal stem cells (DPSCs) were trypsinized (0.25% trypsin EDTA solution (Gibco)) and centrifuged at 2000 rpm 4° C. for 5 minutes. The cell pellets were then used for total RNA extraction, using a suitable extraction kit, High Pure RNA Isolation kit (Roche). The extracted RNA was quantified, and its quality checked by spectrophotometry (Nanodrop ND-1000 Spectrophotometer) in readings from 220 to 350 nm, and then stored at −80° C. until the next step. In further processing, the cDNA was synthesized from the purified RNA, using the Ready-To-Go You-Prime First-Strand Beads kit (GE Healthcare), according to the manufacturer's recommendations. The cDNA was thus synthesized and stored at −20° C. until the next step. At this point, it should be noted that, due to the use of the Oligo (dT) primer, the synthesized cDNA corresponded to the mRNA present in the sample at the time of collection.

The cDNAS synthesized from the DPSCs was evaluated taking into account the expression of six genes, two ‘housekeeping genes’ (β-actin and GAPDH) and four genes encoding stem cell pluripotency markers (c-kit, Oct-4, NANOG and ALP). Primer sequences were taken from the literature, corrected or modified in house and synthesized in an external laboratory (MWG Operon, Germany). Upon arrival, the primers were rehydrated in DNase/RNase free water at a concentration of 100 pmol/μl.

Quantification was performed on a CFX96™ (BioRad) device using the iQ™ SYBR® Green Supermix (BioRad). Each pair of primers corresponding to the gene was used to analyse their expression in the DPSCs cDNA, in duplicate, together with a negative control. The plates containing the mixture corresponding to each of the genes were subjected to the following temperature cycles: 95° C. for 4 minutes, 35 cycles including 95° C. for 20 seconds, 55° C. for 20 seconds and 72° C. for 30 seconds, ending with real-time acquisition and a final extension of 72° C. for 7 minutes. After the temperature cycles, the number corresponding to the Cycle threshold was recorded. The plates containing the amplified genes, or the qPCR products were kept on ice and observed on a 2% agarose gel, to confirm and reinforce the identity of the amplification products. Briefly, 2 g of NuSieve® 3:1 agarose (Lonza) agarose was mixed and dissolved in 100 ml of Tris-Acetate-EDTA buffer by heating, and mixed in ethidium bromide, in a final concentration of 0.2 μg/ml, and poured into a horizontal electrophoresis equipment. After solidification, 15 μl of the qPCR products were added to the agarose wells and subjected to a 120V potential difference for 40 minutes in order to separate the amplification products. The gel was observed under UV light and photographically registered using the GelDoc® 2000 (BioRad) and Quantity One® software (BioRad) software.

### Example 9. Gene Expression of GFAP, NeuN, β-Actin, GAPDH, Nestina, NF-H and GAP-43 of Differentiated DPSCs in Neuroglial Cells

RT-PCR and qPCR was performed at DPSCs after differentiation in neuroglial type cells, to evaluate the gene expression of Glial Fibrillar Acid Protein (GFAP), Nuclear Neuronal Protein (NeuN), β-actin, Glyceraldehyde-3-phosphate dehydrogenase (3GAPDH), Nestina, Heavy Neurofilament (NF-H) and Growth-Associated Protein 43 (GAP-43). The method for performing RT-PCR and qPCR was previously described (at viii).

### Example 10. Differentiation Capacity in Three Lines: Adipogenic, Chondrogenic and Osteogenic

DPSCs isolated and expanded to P3-P5 were tested for their ability to differentiate adipogenic, osteogenic and chondrogenic. In this sense, kits developed for the purpose of Thermo Fisher Scientific were used: StemPro® Osteogenesis Differentiation kit, StemPro® Adipogenesis Differentiation kit and StemPro® Chrondrogenesis Differentiation kit and the manufacturer's instructions must be followed. After differentiation, the samples were processed histologically and stained for analysis in a vertical microscope with phase contrast. In adipogenic, osteogenic and chondrogenic differentiation protocols, cells were washed and fixed with 4% paraformaldehyde for 20 minutes and stained with Oil Red O, Van Kossa and Alcian Blue, respectively.

DPSCs isolated and expanded to P3-P5 were tested for their capacity for neurogenic differentiation. Differentiation in neuroglial cells was achieved by changing the expansion culture medium by means of neurogenic differentiation (Promocell, C-28015). The differentiation medium was maintained until the observation of morphological changes in the DPSCs that confirm their differentiation in neuroglial cells, in an inverted microscope (Zeiss, Germany). The minimum differentiation period was 72 hours.

### Example 11. In Vivo Application of DPSCs in Axonotmesis and Neurotmesis Lesions in the Sasco Sprague Rat Experimental Model

Several resorbable biomaterials associated with isolated DPSCs and expanded to P5 were studied to promote peripheral nerve regeneration after axonotmesis and neurotmesis injuries. DPSCs were used since they have the capacity to produce trophic factors, production of extracellular matrix molecules (ECM), modulation of the local immune and inflammatory response, differentiation in Schwann cells or other cells involved in Wallerian degeneration and axonal regeneration. The biomembranes associated with DPSCs, were used in the reconstruction of a 10 mm continuity solution or after axonotmesis, induced in the rat sciatic nerve. Under general anaesthesia, the sciatic nerve was exposed unilaterally and sectioned immediately above its branch, creating a 10 mm continuity solution. In the case of axonotmesis, a clamp without a serration was used, capable of exerting a force of 54N and which, being pressed for 30 s, results in a perfectly reproducible 3 mm lesion. The application of DPSCs in both types of lesions was done by infiltration (1×10⁵ cells/lesion) at the lesion site and involved by the biomaterial. Morphological studies (optical microscopy and histomorphometry) were carried out after 20 or 12 weeks, in groups of neurotmesis or axonotmesis, respectively.

The functional evaluation of the recovery of the sciatic nerve was performed periodically during this period, through the kinematic analysis of locomotion, the Sciatic Functionality Index (SFI) and the Postural Reaction Extension Extension (RPE). Sensory perception was assessed using the Latency Flexor Reflex (RFL). Each experimental group should consist of 7 animals (N=7) and expanded and isolated DPSCs were used up to 5 teeth in P3-P5 (N=5) whose dental pulp was cryopreserved following this validation protocol.

### Conclusions

It was demonstrated that the method of the present invention herein proposed allows the isolation and expansion of viable DPSCs after thawing, whilst maintaining tissue viability. When the dental pulp is thawed, DPSCs can be isolated from fragment explants, with an average of 8×10⁶ cells after 10 days in culture and using the expansion culture medium.

DPSCs isolated from explants of cryopreserved dental pulp fragments have been shown to have morphological characteristics typical of mesenchymal stem cells according to the criteria established by the ISCT. These DPSCs are able to remain in cell culture and adhere to plastic, forming a confluent monolayer and present the typical phenotype of mesenchymal stem cells.

By cytogenetic analysis performed on the DPSCs of the invention, present no numerical or structural changes in the somatic chromosomes and sex chromosomes.

Additionally, it was demonstrated that these DPSCs do not present neoplastic characteristics and are stable in chromosomal terms (number and structure of somatic and sexual chromosomes) during the isolation and expansion processes.

The obtained DPSCs demonstrated to have differentiation capacity in 4 cell lines: osteogenic, chondrogenic, adipogenic and neurogenic. The ability to differentiate was confirmed by histological analysis, by immunocytochemistry and by RT-PCR.

The DPSCs subcultured up to 3 to 5 passages (P3-P5) present specific markers, a secretome and a metabolic profile characteristic of MSCs.

When DPSCs were applied to subcutaneous implants and to peripheral nerve injuries of axonotmesis and neurotmesis in the rat animal model, they show biocompatibility.

The DPSCs associated with different biomaterials promote the functional and morphological recovery of the peripheral nerve, without originating teratomas or neoplasms.

The lung, kidney, spleen, pancreas, liver collected in model assays with rats (according to ISO 10993-6) present no change induced by the application of DPSCs.

1. A process for preparation and cryopreservation of dental pulp stem cells (DPSCs) from a healthy tooth of a human subject characterized by comprising the following steps:
a. Refrigeration of a provided healthy tooth for less than 72 hours,
b. Washing the tooth for at least 1 time with a sterile compress soaked in an ethyl alcohol solution 70% (V/V),
c. Immersion of the washed tooth in a suitable sterile phosphate buffer solution (DPBS),
d. Disinfection of the washed tooth by immersion in an ethyl alcohol solution 70% (V/V),
e. Drilling one or more holes in the disinfected tooth according to the following procedure: 1-2 transverse and diametrically opposed holes in the neck of incisor and canine teeth, and/or 2-4 transverse and diametrically opposed holes in the neck of premolar or molar teeth, wherein the drill is cooled with a refrigerated sterile phosphate buffer solution (DPBS),
f. Cryopreservation of the drilled tooth by immersion in 7.7 ml vials containing a cryopreservation solution consisting of: 5.6 ml of Culture medium xeno-free, 1, 4 ml of Human Serum Off the Clot Type AB, and 0.7 ml of Dimethyl Sulfoxide (DMSO),
g. Isolation of the dental pulp stem cells (DPSCs) of the tooth treated and cryopreserved as described in the previous steps by thawing followed by an explant procedure, wherein the thawed tooth is immersed in a sterile container containing 100 ml of an expansion culture medium consisting of: 80% (3.5 ml), 80 ml of Culture medium xeno-free and 20% (3.5 ml), 20 ml of Human Serum Off the Clot, Type AB, without heparin to which 0.1 ml of an antibiotic comprising 100 U/ml penicillin, 100 mg/ml streptomycin and 250 μg/ml of amphotericin B is added,
h. Expansion of the isolated of DPSCs obtained in the previous step by growing each sample in the expansion culture medium in a CO₂ incubator at 37° C. for 3 days and further maintaining the culture for at least 7-10 days and replacing the said used culture medium twice a week by a fresh expansion culture medium.

2. A process for preparation and cryopreservation of dental pulp stem cells (DPSCs) according to claim 1 characterized by the tooth being a human definitive tooth.

3. A process for preparation and cryopreservation of dental pulp stem cells (DPSCs) according to claim 1 characterized by the tooth being a human deciduous tooth.

4. A process for preparation and cryopreservation of dental pulp stem cells (DPSCs) according to any of the claim 1 and 2 or 3 characterized by the used tooth being an incisor, canine, premolar and molar teeth, of the mandibular arch, maxillary arch and included tooth.

5. An isolate of dental pulp stem cells (DPSCs) characterized by being obtainable by the process as described in claims 1 to 4.

6. An isolate of dental pulp stem cells (DPSCs) according to claim 5 characterized by the DPSCs present after isolation and expansion up to sub-culturing passage P3-P5:
morphological characteristics typical of mesenchymal stem cells (MSCs),
specific immunocytochemical markers characteristic of MSCs: NANOG, Stro-1, c-kit, CD31, vimentin, CD34, CD 45, CD73, CD90 and CD105 markers,
a secretome and a metabolic profile characteristic of MSCs,
no numerical or structural changes in the somatic chromosomes and sex chromosomes with reference to the source DPSCs,
no neoplastic characteristics with reference to the source DPSCs,
stability in chromosomal terms with reference to the source DPSCs,
capacity to differentiate in 4 cell lines: osteogenic, chondrogenic, adipogenic and neurogenic lines.

7. An isolate of dental pulp stem cells (DPSCs) according to claim 5 or 6 characterized by DPSCs the neuroglial cells present after differentiation in neurogenic lines, GFAP, GAP-43 and NeuN markers and express genes GFAP, NeuN, β-actin, GAPDH, Nestina, NF-H and GAP-43.

8. An isolate of dental pulp stem cells (DPSCs) as described in any of the claims 5 to 7 characterized for as a medicament.

9. An isolate of dental pulp stem cells (DPSCs) according to claim 8 characterized for use in regenerative therapy.

10. An isolate of dental pulp stem cells (DPSCs) according to any of the claim 8 or 9 characterized for use in bone, nervous, vascular, musculoskeletal regenerative therapy.

11. An isolate of dental pulp stem cells (DPSCs) according to any of the claims 8 to 10 characterized for use peripheral nerve: regeneration therapy after axonotmesis and neurotmesis injuries.

12. An isolate of dental pulp stem cells (DPSCs) according to any of the claims 8 to 11 characterized for use as subcutaneous implants.
